(19) United States
(12) Patent Application Publication
(10) Pub. No.: US 2008/0065088 A1
(43) Pub. Date: Mar. 13, 2008

(54) Bone Cement Mixing Systems and Related Methods

(75) Inventors: Martin Hughes, Wavendon (GB); Darren Seymour, Huntingdon (GB); Colin Turner, Girton (GB); Paul Harmer, Comberton (GB); Richard Cornell, Woburn, MA (US); Howard Seeherman, Cambridge, MA (US)

Correspondence Address: FISH & RICHARDSON PC, P.O. BOX 1022, MINNEAPOLIS, MN, 55440-1022, US

(73) Assignee: WYETH, Madison, NJ (US)

(21) Appl. No.: 11/846,625

(22) Filed: Aug. 29, 2007

Related U.S. Application Data
(60) Provisional application No. 60/842,751, filed on Sep. 7, 2006.

(51) Int. Cl. A61B 17/58; A61M 31/00; A61M 5/178

(52) U.S. Cl. 606/93; 604/38; 604/513; 606/94

(57) ABSTRACT

Bone cement mixing systems and related methods are disclosed. The bone cement mixing systems can include a first chamber, a second chamber, and a passage fluidly connecting the first and second chambers. A first piston can be disposed in the first chamber, and a second piston can be disposed in the second chamber.

### CROSS-REFERENCE TO RELATED APPLICATIONS

This application claims the benefit of U.S. Application Ser. No. 60/842,751, filed on Sep. 7, 2006.

### TECHNICAL FIELD

This invention relates to bone cement mixing systems and related methods.

### BACKGROUND

Bone cements, such as calcium phosphate based bone cements, can be used during certain medical treatments to help repair and/or reconstruct bone (e.g., fractured bone). The ability of certain bone cements to repair and/or reconstruct bone can be enhanced by the inclusion of recombinant human bone morphogenetic protein (rhBMP-2), which promotes the growth of bone. An example of a calcium phosphate based bone cement enhanced in this manner is rhBMP-2/CPM.

To prepare bone cements, such as calcium phosphate based bone cements, a powdery substance is generally combined with a liquid, and the resultant combination is mixed together to form a bone cement paste. The bone cement paste can then be delivered to a treatment site (e.g., a fracture site) to help repair and/or reconstruct the bone.

### SUMMARY

In one aspect of the invention, a bone cement mixing system includes a housing defining a first chamber, a second chamber, and a passage fluidly connecting the first and second chambers. A first piston is slidably disposed within the first chamber, and a second piston is slidably disposed within the second chamber. A bone cement delivery device is disposed within the second chamber. The bone cement delivery device defines a third chamber and is adaptable to place the third chamber in fluid communication with the first chamber.

In another aspect of the invention, a system includes a housing defining a first chamber, a second chamber, and a passage fluidly connecting the first and second chambers. The housing is configured so that a liquid injection device can be secured thereto. The liquid injection device is in fluid communication with at least one of the first and second chambers when secured to the housing. The system also includes a first piston slidably disposed within the first chamber and a second piston slidably disposed within the second chamber. A bone cement delivery device is disposed in the second chamber.

In an additional aspect of the invention, a method includes passing a bone cement paste through a first passage that fluidly connects a first chamber and a second chamber. The first passage is configured to cause a first level of shear within the bone cement paste as the bone cement paste is passed therethrough. The method further includes passing the bone cement paste through a second passage that fluidly connects the first chamber to a third chamber. The second passage is configured to cause a second level of shear as the bone cement paste is passed therethrough. The second level of shear is different than the first level of shear.

Embodiments can include one or more of the following features.

In some embodiments, the bone cement delivery device is disposed within a bore in the second piston.

In certain embodiments, the bone cement delivery device includes an axially displaceable pin arranged to fit within an aperture in a seal of the second piston such that there is substantially no fluid communication between the first chamber of the housing and the third chamber of the bone cement delivery device when the pin is disposed within the bore in the seal of the second piston.

In some embodiments, the pin is capable of being retracted from the aperture in the seal of the second piston, and the first chamber of the housing is in fluid communication with the third chamber of the bone cement delivery device when the pin is retracted from the aperture in the seal of the second piston.

In certain embodiments, the passage that fluidly connects the first and second chambers has a reduced cross-sectional area relative to the first and second chambers.

In some embodiments, a passage that fluidly connects the first and third chambers when the third chamber is placed in fluid communication with the first chamber has a reduced cross-sectional area relative to the first and third chambers.

In some embodiments, the bone cement mixing system includes a bone cement powder disposed within at least one of the first and second chambers.

In certain embodiments, the bone cement powder is an osteoconductive powder (e.g., a calcium phosphate based bone cement powder, such as a calcium phosphate/sodium bicarbonate blended powder).

In some embodiments, the bone cement powder forms a bone cement paste when a liquid is added to the bone cement powder.

In certain embodiments, the bone cement paste can be mixed by axially displacing the first and second pistons within the first and second chambers, respectively.

In some embodiments, the liquid includes bone morphogenetic protein (e.g., recombinant human bone morphogenetic protein, such as rhBMP-2).

In certain embodiments, the bone cement delivery device is slidably disposed within the second chamber.

In some embodiments, the bone cement delivery device includes a syringe.

In certain embodiments, the syringe includes a fitting (e.g., a Luer Lock fitting) configured to secure the syringe to the second piston.

In some embodiments, the passage is partially formed by a mixing post and a mixing anvil extending from an inner surface of the housing.

In certain embodiments, the housing includes a fitting configured to allow a liquid injection device to be secured thereto.

In some embodiments, the liquid injection device is in fluid communication with the first and second chambers when secured to the inlet fitting.

In certain embodiments, the bone cement delivery device includes a tube and circumferentially spaced ribs extending from the tube. The circumferentially spaced ribs are arranged to cooperate with the second piston to form channels configured to permit gases to pass therethrough.

In some embodiments, the bone cement mixing system further includes a porous membrane disposed over a region of the bone cement delivery device that defines at least one aperture.

In certain embodiments, the system (e.g., the bone cement mixing system) is a single use system (e.g., a single use bone cement mixing system).

In some embodiments, a liquid injection device is secured to the housing.

In certain embodiments, a bone cement powder is disposed within at least one of the first and second chambers, and the bone cement powder forms a bone cement paste when a liquid is transferred from the liquid injection device into the at least one of the first and second chambers.

In some embodiments, the first and second pistons are capable of passing bone cement paste back and forth between the first and second chambers when the first and second pistons are alternately depressed.

In certain embodiments, the bone cement delivery device defines a third chamber, and the bone cement delivery device is adaptable to place the third chamber in fluid communication with the first chamber.

In some embodiments, the first piston and a plunger of the bone cement delivery device are capable of passing bone cement paste back and forth between the first and third chambers when the first piston and the plunger are alternately depressed and the third chamber of the bone cement delivery device is in fluid communication with the first chamber.

In some embodiments, the third chamber is formed by a bone cement delivery device disposed in the second chamber.

In certain embodiments, the method further includes removing the bone cement delivery device from the second chamber after passing the bone cement paste into the third chamber.

In some embodiments, passing the bone cement paste through the first passage imparts a first level of shear to the bone, cement paste and passing the bone cement paste through the second passage imparts a second level of shear to the bone cement paste, and the first level of shear is lower than the second level of shear.

In certain embodiments, the method includes passing the bone cement paste through the first passage prior to passing the bone cement paste through the second passage.

In some embodiments, the method further includes introducing a liquid into at least one of the first and second chambers.

Embodiments can include one or more of the following advantages.

In some embodiments, the bone cement mixing system permits bone cement paste to be thoroughly mixed. Using the bone cement mixing system, for example, the mixing can be carried out in two stages. In the first stage, the paste is subjected to a relatively low level of shear (e.g., by being repeatedly forced past an obstruction). In the second stage, the paste is subjected to a relatively high level of shear (e.g., by being repeatedly forced through a smaller orifice). Thoroughly mixing the bone cement paste can help to improve the injectability of the bone cement paste. Thoroughly mixing the bone cement paste can, for example, reduce (e.g., minimize) the possibility of filter pressing, which occurs when liquid constituents of the bone cement paste pass through solid constituents of the bone cement paste during injection, leaving a solid uninjectable mass behind.

In certain embodiments, the efficiency of mixing bone cement paste in the bone cement mixing system is increased. By splitting the mixing into two stages, for example, it is possible to achieve a thorough mixing of the cement paste within a short time and with a reduced amount of physical effort. During the first mixing stage, for example, the bone cement paste can be passed back and forth between mixing chambers via a passage having a relatively large cross-sectional area. This can help to reduce the amount of physical effort required to initially mix the bone cement paste, which can be relatively dry and difficult to mix in the initial phases of mixing. During the second mixing stage, the bone cement paste can be passed back and forth between mixing chambers via a passage having a smaller cross-sectional area. This can increase the levels of shear within the bone cement paste to provide more thorough mixing. Due to the increased wetness of the bone cement paste during the second stage of mixing, the bone cement paste can be passed through the passage of reduced cross-sectional area without an excessive amount of physical effort.

In some embodiments, the mixing chamber of the bone cement mixing system is fluid tight (e.g., gas tight). As a result, during mixing of the bone cement paste, a volume of gas can become incorporated within the bone cement paste, significantly reducing the effort required to mix and subsequently inject the bone cement paste.

In certain embodiments, the bone cement mixing system helps to reduce the amount of bone cement paste remaining in the bone cement mixing system at the end of the mixing process. This can help to reduce the loss of expensive drug contents during the mixing and delivery process.

In some embodiments, the bone cement mixing system allows for relatively easy transfer of the bone cement paste from a mixing chamber of the system to a bone cement delivery device. The bone cement delivery device can, for example, be a component of the bone cement mixing system, allowing the bone cement paste to be transferred from one portion of the bone cement mixing system (e.g., from a mixing chamber of the bone cement mixing system) to the bone cement delivery device with little effort. After mixing the bone cement paste, substantially all of the bone cement paste can be disposed within the bone cement delivery device, which can then be removed from the remainder of the bone cement mixing system.

In some embodiments, the risk of contamination of the bone cement paste and the ingredients of the bone cement paste can be reduced. The bone cement paste and its ingredients can, for example, be retained within the bone cement mixing system or a liquid injection device (e.g., a syringe) throughout the mixing procedure, thereby reducing the risk of contamination to the bone cement paste.

In certain embodiments, the bone cement mixing system is constructed for single use and/or is disposable. The bone cement mixing system can be relatively inexpensive and easy to use.

Other aspects, features, and advantages will be apparent from the description and drawings, and from the claims.

### DESCRIPTION OF DRAWINGS

FIG. 1 is a perspective view of a bone cement mixing system.

FIG. 2 is a cross-sectional view of the bone cement mixing system of FIG. 1.

FIG. 3 is a perspective, partial cut-away view of the bone cement mixing system of FIG. 1.

FIGS. 4A-4H illustrate a method of using the bone cement mixing and delivery device of FIG. 1.

### DETAILED DESCRIPTION

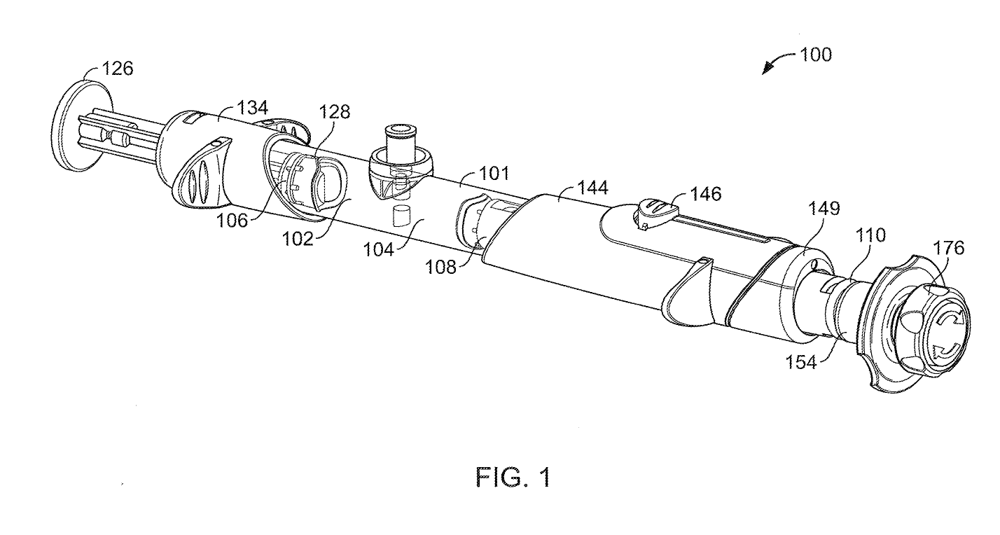

Referring to FIGS. 1-3, a bone cement mixing system 100 includes a housing 101 that has first and second mixing chambers 102 and 104. A first piston 106 is disposed within first mixing chamber 102, and a second piston 108 is dispose a within second mixing chamber 104. A bone cement delivery device (e.g., a syringe) 110 is disposed within an axial bore 112 formed in second piston 108. Bone cement delivery device 110 includes a mixing/delivery chamber 114 extending axially along its length and a plunger 115 disposed within mixing/delivery chamber 114. First piston 106 is arranged to slide axially within first mixing chamber 102, and the assembly of second piston 108 and bone cement delivery device 110 is arranged slide axially within second mixing chamber 104. Similarly, plunger 115 is arranged to slide axially within mixing/delivery chamber 114 of bone cement delivery device 110.

During use, as discussed below, a bone cement paste is contained within first mixing chamber 102 and/or second mixing chamber 104. Bone cement mixing system 100 can be used to mix the bone cement paste in a two stage mixing process. In the first stage, the bone cement paste is transferred back and forth between first and second mixing chambers 102 and 104 by alternately sliding first and second pistons 106 and 108 within first and second mixing chambers 102 and 104, respectively. In the second stage, the bone cement paste is transferred back and forth between first mixing chamber 102 and mixing/delivery chamber 114 of bone cement delivery device 110 by sliding first piston 106 and plunger 115 back and forth within first mixing chamber 102 and mixing/delivery chamber 114, respectively. The bone cement mixing system 100 can be configured so that the second stage of mixing imparts higher levels of shear to the bone cement paste than the first stage of mixing. This can help to ensure that the bone cement paste is thoroughly mixed during the mixing process and can help to increase the ease with which the user is able to mix the bone cement paste. After thoroughly mixing the bone cement paste, substantially all of the bone cement paste can be transferred into mixing/delivery chamber 114 of bone cement delivery device 110, and bone cement delivery device 110 can be removed from axial bore 112 of second piston 108. Bone cement delivery device 110 can then be used to carry out a medical treatment. For example, bone cement delivery device 110 can be used to inject the bone cement paste into a treatment, site (e.g., a bone fracture site) of a patient.

Housing 101, as shown in FIGS. 1-3, is a generally tubular member that includes first and second mixing chambers 102 and 104. First and second mixing chambers 102, 104 of housing 101 can have a diameter of about 6 mm to about 20 mm (e.g., about 10 mm to about 12 mm, about 11 mm), and can have a length of about 30 mm to about 70 mm (e.g., about 40 mm to about 60 mm, about 50 mm). In some embodiments, first and second mixing chambers 102, 104 each have a volume of about 1 ml to about 10 ml (e.g., about 3 ml to about 7 ml). Housing 101 can be formed of one or more materials, such as plastics, metals (e.g., corrosion resistant metals), ceramics, or glasses. Housing 101 can be formed using one or more techniques, such as injection molding techniques, extrusion techniques, machining techniques.

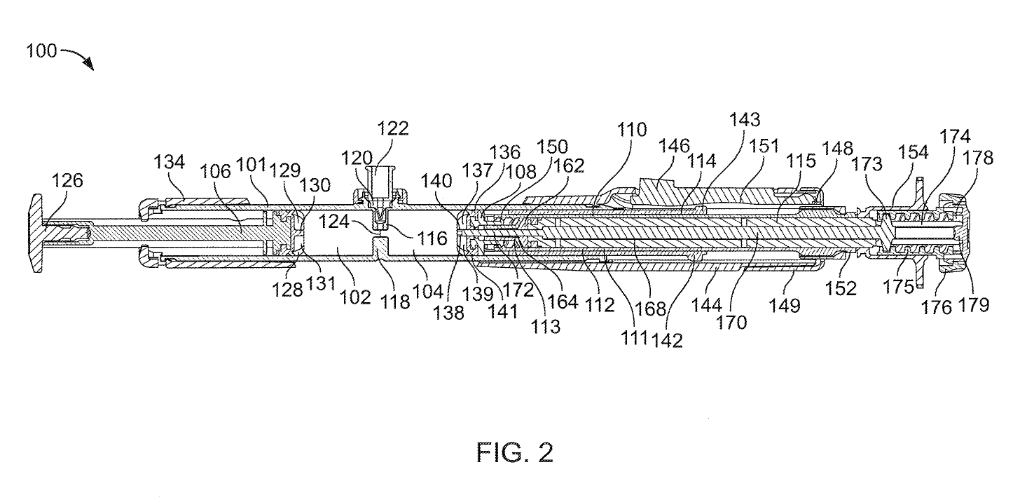

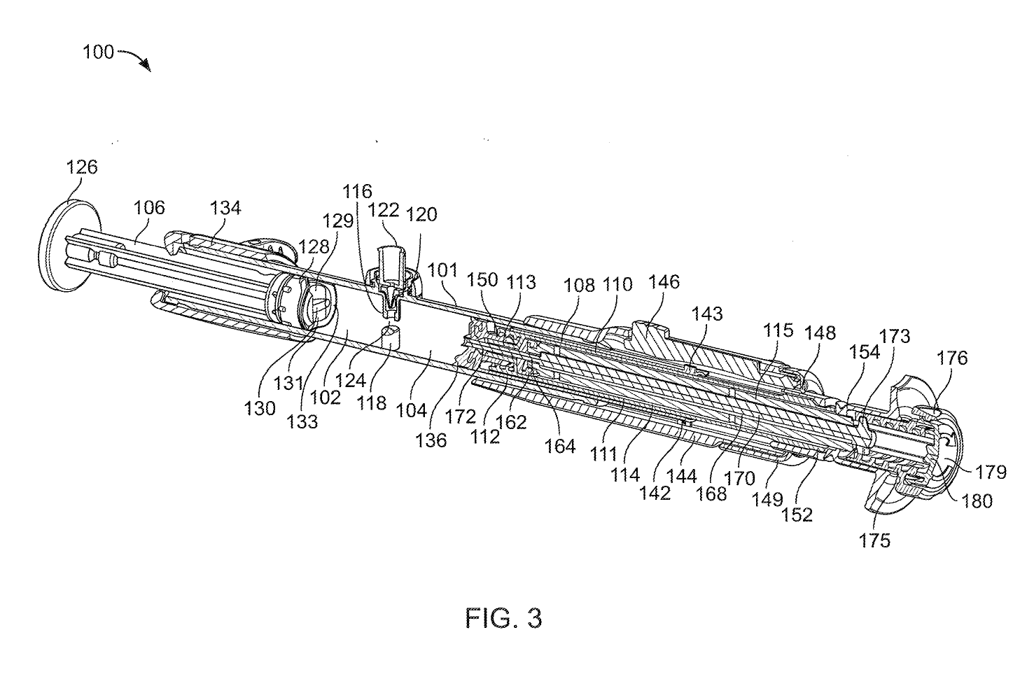

Referring to FIGS. 2 and 3, a mixing post 116 and a mixing anvil 118 extend inwardly from an inner surface of housing 101, between first and second mixing chambers 102 and 104. In some embodiments, mixing post 116 is a substantially cylindrical or frustro-conical member that extends inwardly from the inner surface of housing 101. Mixing post 116 can alternatively or additionally be formed in other shapes. In certain embodiments, for example, mixing post 116 has a circular, elliptical, diamond shaped, and/or triangular cross section. Mixing post 116 typically has a length slightly less than half the diameter of mixing chambers 102 and 104. In some embodiments, mixing post 116 has a diameter (e.g., a base diameter) of about 3 mm to about 6 mm. Mixing post 116 includes a bore extending therethrough that leads to the interior of housing 101. In some embodiments, mixing post 116 is integrally molded with housing 101. Alternatively, mixing post 116 can be a separate member that is attached (e.g., bonded, adhesively attached, etc.) to the inner surface of housing 101.

A one-way valve 120 is fitted within the bore in mixing post 116. One-way valve 120 allows liquid and/or gas to pass into bone cement mixing system 100 (e.g., into first and second mixing chambers 102 and 104 of bone cement mixing system 100), but prevents liquid and/or gas from passing out of bone cement mixing system 100. An inlet fitting 122 retains one-way valve 120 within the bore in mixing post 116. Inlet fitting 122 is secured to housing 101 and is configured to allow a device for injecting liquid (e.g., a syringe) to be secured thereto. Inlet fitting 122 can, for example, include a Luer Lock taper to allow connection to a conventional syringe. When a syringe is not secured to inlet fitting 122, a cap can be secured to inlet fitting 122. The cap, along with one-way valve 120, can help to prevent liquid and/or gases from exiting first and second mixing chambers 102, 104 of housing 101.

In some embodiments, mixing anvil 118 has a shape similar to that of mixing post 116. Mixing anvil 118 can, for example, be a substantially cylindrical or frustro-conical member that extends inwardly from the inner surface of housing 101. However, mixing anvil 118 can alternatively be formed in other shapes. In some embodiments, for example, mixing anvil 118 has a circular, elliptical, diamond shaped, and/or triangular cross section. In certain embodiments, mixing anvil 118 is a substantially solid member. Mixing anvil 118 can alternatively be a hollow member. Mixing anvil 118 typically has a length slightly less than half the diameter of mixing chambers 102 and 104. As a result, a gap generally exists between opposed surfaces of mixing post 116 and mixing anvil 118. In certain embodiments, mixing anvil has a diameter (e.g., a base diameter) of about 3 mm to about 6 mm. In some embodiments, mixing anvil 118 is integrally molded with housing 101. Alternatively, mixing anvil 118 can be a separate member that is attached (e.g., bonded, adhesively attached, etc.) to the inner surface of housing 101.

Still referring to FIGS. 2 and 3, a passage 124 extends between first mixing chamber 102 and second mixing chamber 104 and fluidly connects first mixing chamber 102 to second mixing chamber 104. Passage 124 is formed by mixing post 116, mixing anvil 118, and the inner surface of housing 101. Due to the obstruction caused by mixing post 116 and mixing anvil 18, passage 124 has a reduced cross-sectional area relative to mixing chambers 102 and 104. For example, passage 124 can have a cross-sectional area that is at least about 40 percent less (e.g., at least about 50 percent less, at least about 60 percent less, at least about 70 percent less) than the cross-sectional areas of mixing chambers 102 and 104 and/or at most about 80 percent less (e.g., at most about 70 percent less, at most about 60 percent less, at most about 50 percent less) than the cross-sectional areas of mixing chambers 102 and 104.

Referring now to FIGS. 1-3, first piston 106 is disposed in first mixing chamber 102. First piston 106 is an elongate member having a cruciform cross-section. First piston 106 can have a length greater than or equal to the length of first chamber 102. In some embodiments, first piston 106 includes, (e.g., is formed of) one or more polymeric materials, such as polycarbonates, polysulfones, acetals, polyamides, polyethylenes, polypropylenes, polyesters, polylurethanes, ABS, PVDF, PET, PBT, liquid crystal polymers or PTFE. Alternatively or additionally, first piston 106 can include (e.g., can be formed of) one or more other materials, such as metals (e.g., stainless steels, aluminums, or brasses), ceramics, and/or rubbers.

A head 126 of enlarged diameter is secured to an end region of first piston 106. Head 126 can facilitate pushing of first piston 106 inward and/or pulling of first piston 106 outward during use. Head 126 can be secured to first piston 106 using any of various techniques. For example, head 126 can be secured to first piston 106 by an interference friction fit, snap fit, adhesive, and/or a screw thread. Alternatively or additionally, head 126 can be integrally formed with first piston 106.

A resilient seal 128 is provided at the end of first piston 106 opposite head 126. As shown in FIGS. 2 and 3, seal 128 is a substantially cylindrical member with two recesses 129 and 131 formed in its front face. Recesses 129 and 131 are shaped to receive mixing post 116 and mixing anvil 118, respectively, such that seal 128 conforms to (e.g., fits around) mixing post 116 and mixing anvil 118 when first piston 106 is fully inserted into first mixing chamber 102. A web or rib 130 extends between recesses 129 and 131. Due to the shape of seal 128, when first piston 106 is pushed all the way into first chamber 102, recesses 129 and 131 mate with the mixing post 116 and mixing anvil 118, respectively, and rib 130 becomes disposed between mixing post 116 and mixing anvil 118. Seal 128 also includes a flexible lip 133 that extends about the circumference of the front face and contacts the inner surface of housing 101.

Seal 128 can be sized and shaped such that a substantially fluid-tight seal is created between seal 128 and the inner surface of the portion of housing 101 that forms first mixing chamber 102. Seal 128 can, for example, have an outer diameter that is about 0.1 mm to 0.5 mm greater than the inner diameter of the portion of housing 101 that forms first mixing chamber 102. The fluid-tight seal can be enhanced by flexible lip 133, which is held in contact with the inner surface of housing 101. Increasing fluid pressure within first mixing chamber 102 will press lip 133 into firm contact with the inner surface of housing 101 thereby improving the integrity of the seal. The fluid-tight seal can prevent bone cement paste and/or gases from flowing around seal 128 during the cement mixing process.

Seal 128 can include (e.g., can be formed of) one or more resilient materials, such as injection moldable or compression moldable plastic elastomers, rubbers, or silicone rubbers. In some embodiments seal 128 includes a relatively non-resilient core surrounded by a resilient coating. In some embodiments, seal 128 is formed separately from first piston 106 and then attached to first-piston 106. Alternatively, seal 128 and first piston 106 can be formed integral with one another by such processes as over-molding or two shot molding.

Referring to FIGS. 1-3, first piston 106 is substantially prevented from rotating within housing 101 by a cap 134 that is secured to housing 101. Cap 134 can be secured to housing 101 using a snap fitting technique. Cap 134, for example, includes one or more snaps that project into a recess formed in the outer surface of housing 101 when cap 134 is slid onto housing 101. This arrangement secures cap 134 in a fixed axial position relative to housing 101. Cap 134 can be prevented from rotating relative to housing 101 by a series of projections (e.g., castellations) extending from its inner surface that mate with corresponding projections (e.g., castellations) on an opposed end face of housing 101. Alternatively or additionally, any of various other techniques can be used to secure cap 134 to housing 101 in an axially and rotationally fixed arrangement, such as frictional interference, adhesive, threads and/or mechanical fasteners. Cap 134 includes a cruciform slot that receives and engages the cruciform shaft of first piston 106 with a clearance allowing free axial movement of piston 106. Cap 134, however, substantially prevents first piston 106 from rotating relative to housing 101 and cap 134.

As shown in FIGS. 2 and 3, second piston 108 is disposed within second mixing chamber 104. Second piston 108 is an elongate, substantially cylindrical member through which axial bore 112 extends. Axial bore 112 is sized and shaped to receive bone cement delivery device 110 therein. Second piston 108 can include (e.g., can be formed of) one or more, polymeric materials, such as polycarbonates, polysulfones, acetals, polyamides, polyethylenes, polypropylenes, polyesters, polyurethanes, ABS, PVDF, PET, PBT, liquid crystal polymers, and/or PTFE. Alternatively or additionally, second piston 108 can include (e.g., can be formed of) one or more other materials, such as metals (e.g., stainless steels, aluminums, or brasses), ceramics, and/or rubbers.

A resilient seal 136 is provided at an end region of second piston 108. Seal 136 is a substantially cylindrical member with two recesses 137 and 139 formed in its front face. Recesses 137 and 139 are shaped to receive mixing post 116 and mixing anvil 118, respectively, such that seal 136 conforms to (e.g., fits around) mixing post 116 and mixing anvil 118. Due to the shape of seal 136, when second piston 108 is pushed all the way into second chamber 104, recesses 137 and 139 mate with mixing post 116 and mixing anvil 118, respectively. Seal 136 also includes a rib 140 that extends between recesses 137 and 139 and a flexible lip 141 that extends about the circumference of the front face and contacts the inner surface of housing 101.

Seal 136 includes a central aperture 138 extending axially therethrough. Aperture 138 can have a diameter of about 11.0 mm to about 2.5 mm (e.g., about 1.9 mm). Seal 136 is sized and shaped such that a substantially fluid-tight seal is created between seal 136 and the inner surface of the portion of housing 101 that forms second mixing chamber 104. Seal 136 can, for example, have an outer diameter that is about 0.1 mm to about 0.5 mm greater than the inner diameter of the portion of housing 101 that forms second mixing chamber 104. The fluid-tight seal can be enhanced by flexible lip 141, which is held in contact with the inner surface of housing 101. Increasing fluid pressure within second mixing chamber 104 will press lip 141 into firm contact with the inner surface of housing 101 thereby improving the integrity of the seal.

Seal 136 can include (e.g., can be formed of) one or more resilient materials, such as injection moldable or compression moldable plastic elastomers, rubbers, and/or silicone rubbers. In some embodiments, seal 136 includes a core of a relatively non-resilient material and a coating of a relatively resilient material. In some embodiments, seal 136 is formed separately from second piston 108 and then attached to second piston 106. Alternatively, seal 136 and second piston 108 can be formed integral with one another by such processes as over-molding or two shot molding.

Second piston 108 is a tubular member that includes circumferentially spaced ribs 142 extending from its outer surface in an end region opposite seal 136. Ribs 142 of second piston 108 can help to prevent rotation of second piston 108 relative to housing 101 and can allow bone cement delivery device 110 to be rotated relative to second piston 108 in order to remove bone cement delivery device 110 from second piston 108 at the end of the mixing process, as discussed below. A block 143 also extends radially outward from the end region of second piston 108 opposite seal 136. Block 143 can, for example, extend radially outward between two adjacent ribs 142.

Referring to FIGS. 1-3, an extended end cap 144 is keyed to housing 101. Extended end cap 144 can, for example include radial projections that mate with matching cut-outs in the end face of housing 101. As a result, rotation of extended end cap 144 relative to housing 101 can be reduced or prevented. Any of various alternative techniques, such as snap fitting, bonding, adhesive attachment, etc., can alternatively or additionally be used to help prevent extended end cap 144 from rotating relative to housing 101. Extended end cap 144 carries radial inwardly extending ribs that define longitudinal slots in which ribs 142 of second piston 108 are received when extended end cap 144 is slid onto housing 101. As a result of this arrangement, rotation of second piston 108 within housing 101 is reduced or prevented by extended end cap 144.

A lever 146 is retained within an aperture formed in the wall of extended end cap 144. When second piston 108 is slid fully into second mixing chamber 104, block 143 of second piston 108 is disposed at a location between lever 146 and housing 101. Lever 146 is secured at one end to a lever end cap 149. In particular, as shown in FIGS. 2 and 3, an end region of lever 146 is disposed within a cavity formed by a platform 148 that extends integrally from an inner surface of lever end cap 149. Lever end cap 149 is retained on extended end cap 144 by mechanical snaps. Alternatively or additionally lever end cap 149 can be retained on extended end cap 144 using other fastening techniques, such as adhesive, frictional interference, or mechanical fasteners. Lever 146 is retained in position within the aperture by mechanical fasteners, such as snaps. These mechanical fasteners are arranged such that their retaining force can be overcome by finger pressure (approximately 15 N to 20 N) on lever 146.

Still referring to FIGS. 2 and 3, when second piston 108 is slid fully into second mixing chamber 104, block 143 is located between lever 146 and housing 101. In this position, lever 146 can be pressed radially inwards. Once pressed radially inward, the mechanical fasteners (e.g., snaps) of lever 146 prevent the lever from moving radially outward-unless a substantial radial outward force is applied to lever 146. With lever 146 in this inwardly depressed position, second piston 108 is prevented from sliding axially within second mixing chamber 104 due to contact between the end surface of lever 146 and block 143. Should the user attempt to press lever 146 radially inwards prior to fully inserting second piston 108 into second mixing chamber 104, then block 143 will come into contact with a ramp 151 on the lower face of lever 146. Subsequent inward movement of second piston 108 and thus block 143 into second mixing chamber 104 will apply a radially outward force to lever 146, causing lever 146 to be lifted back into its former position in extended end cap 144.

When second piston 108 is slid fully into second mixing chamber 104, seal 136 is adjacent passage 124 formed between mixing post 116 and anvil 118. As discussed above, recesses 129 and 131 of seal 128 receive mixing post 116 and mixing anvil 118, respectively, therein and rib 130 of seal 128 fits between the opposed faces of mixing post 116 and mixing anvil 118 when first piston 106 is slid fully into first mixing chamber 102. Similarly, when second piston 108 is slid fully into second mixing chamber 104, recesses 137 and 139 receive mixing post 116 and mixing anvil 118, respectively, and rib 140 fits between mixing post 116 and mixing anvil 118. As a result, when first and second pistons 106 and 108 are displaced fully toward the central portion of housing 101, front faces of seals 128 and 136 can contact one another.

Bone cement delivery device 110 can be disposed within axial bore 112 of second piston 108 and secured to second piston 108 via a Luer Lock taper 150 of second piston 108. Bone cement delivery device 110 includes a tubular body portion 111 and a tapered tip 113 of reduced diameter extending from a distal end of tubular body portion 111. A series of fine axial grooves 152 are formed on the inner surface of body portion 111 in an end region of body portion 111 opposite tapered tip 113. Gases can escape bone cement delivery device 110 via axial grooves 152 during use, as discussed below.

As shown in FIGS. 1-3, bone cement delivery device 110 includes an extension cap 154 that is secured to tubular body portion 11. Extension cap 154 can, for example, include snaps that project into voids formed in the outer surface of tubular body portion 111 in order to secure extension cap 154 to tubular body portion 111. Alternatively or additionally, other attachment techniques, such as bonding, adhesive, etc., can be used to secure extension cap 154 to tubular body portion 111. Extension cap 154 can alternatively be integrally formed with tubular body portion 111. Extension cap 154 provides the user with a member to grip when pushing and pulling the assembly of second piston 108 and bone cement delivery device 110 within second mixing chamber 104.

Referring to FIGS. 2 and 3, plunger 115 is disposed within mixing/delivery chamber 114 of bone cement delivery device 110. A resilient seal 162 is secured to an end region of plunger 115. Seal 162 includes a central aperture 164 extending axially therethrough. Aperture 164 can have a diameter of about 1.9 mm to about 2.1 mm (e.g., about 2.0 mm). Seal 162 can be sized and shaped such that a substantially fluid-tight seal is created between seal 162 and the inner surface of the portion of bone cement delivery device 110 that forms mixing/delivery chamber 114. Seal 162 can, for example, have an outer diameter that is about 0.1 mm to 0.5 mm greater than the inner diameter of the portion of bone cement delivery device 110 that forms mixing/delivery chamber 114. Seal 162 can include (e.g., can be formed of) one or more resilient materials, such as injection moldable or compression moldable plastic elastomers, rubbers, and/or silicone rubbers. In some embodiments, seal 162 includes a core of a relatively non-resilient material and a coating of a relatively resilient material. In some embodiments, seal 162 is formed separately from plunger 115 and then attached to plunger 115. Alternatively, seal 162 and plunger 115 can be formed integral with one another by such processes as over-molding or two shot molding.

Plunger 115 includes a central bore 168 extending therethrough. A plunger shaft 170 is disposed within central bore 168 and is configured to slide axially within central bore 168. A pin 172 is secured to an end region of plunger shaft 170. Pin 172 can be secured to plunger shaft 170 by, for example, adhesive, interference fit, or insert molding. Alternatively, pin 172 can be an integral part of plunger shaft 170. Pin 172 is sized and shaped to pass through apertures 138 and 164 of seals 136 and 162, respectively. Pin 172 can, for example, be sized and shaped to form a fluid-tight seal (e.g., a gas-tight seal) with seals 136 and 162 when disposed in apertures 138 and 164. In certain embodiments, pin 172 has an outer diameter that is substantially equal to the diameters of apertures 138 and 164. Alternatively, the diameter of pin 172 can be slightly larger than the diameters of apertures 138 and 164. In some embodiments, pin 172 has an outer diameter of about 1.9 mm to about 2.1 mm.

Lugs 173 extend radially from the proximal end of plunger shaft 170. Lugs 173 engage a thread 175 of a rotary cap 176. A portion of rotary cap 176 is rotatably disposed within a bore of extension cap 154. Plunger 115 carries at its proximal end ridges 178 that engage with the proximal face of rotary cap 176. Thus, rotary cap 176 can rotate freely about plunger 115 but is restrained against axial movement relative to plunger 115 by ridges 178 and a retainer cap 179. Retainer cap 179 carries a central boss 180 which fits within bore 168 of plunger 115 to hold ridges 178 radially outwards. When rotary cap 178 is rotated relative to extension cap 154 and plunger 115, lugs 173 are drawn through slots 174 in plunger 115 by action of thread 175. This draws plunger shaft 170 and pin 172 in a proximal direction relative to plunger 115. When plunger shaft 170 is pulled outward to its full extent (e.g., pulled outward until lugs 173 contact the end of thread 175), pin 172 is displaced out of aperture 138 in seal 136, putting first mixing chamber 102 in fluid communication with mixing/delivery chamber 114 of bone cement delivery device 110.

If rotary cap 176 is pulled outward, plunger 115 is also drawn outward by ridges 178. Seal 162 is thus also drawn along mixing/delivery chamber 114. When plunger 115 is pulled fully outwards, seal 162 comes into radial contact with grooves 152 in body portion 111. Grooves 152, in combination with the outer diameter of seal 162, form a series of fine axial channels which allow the passage of gas (but, not liquid or paste) out of mixing/delivery chamber 114.

Prior to use, bone cement mixing system 100 is supplied with dry calcium phosphate/sodium bicarbonate blended powder (CPM) tightly packed into mixing chambers 102 and 104 between seals 128 and 136 of first and second pistons 106 and 108, respectively. The powder can, for example, be disposed within first mixing chamber 102 and/or second mixing chamber 104 during assembly of bone cement mixing system 100. The powder can be tightly packed such that there is substantially no free space (e.g., substantially no air or gas pockets) in the powder volume. The tight packing of the powder can help to ensure that liquid injected into the powder to form bone cement paste, as described below, wicks substantially evenly throughout the powder body. In some embodiments, the powder is equally distributed on either side of the mixing post 116 and mixing anvil 118. For example, substantially equal amounts of powder can be disposed in first mixing chamber 102 and second mixing chamber 104. For a nominal device output of about 3 ml, the distance between seal 128 and seal 136 can be about 40 mm or less.

Bone cement mixing system 100 can be supplied in a restraining tray (not shown) which presents bone cement mixing system 100 in a substantially horizontal position with inlet fitting 122 extending upwardly. The restraining tray can be constructed to restrain first and second pistons 106 and 108 against outward movement away from the center of bone cement mixing system 100.

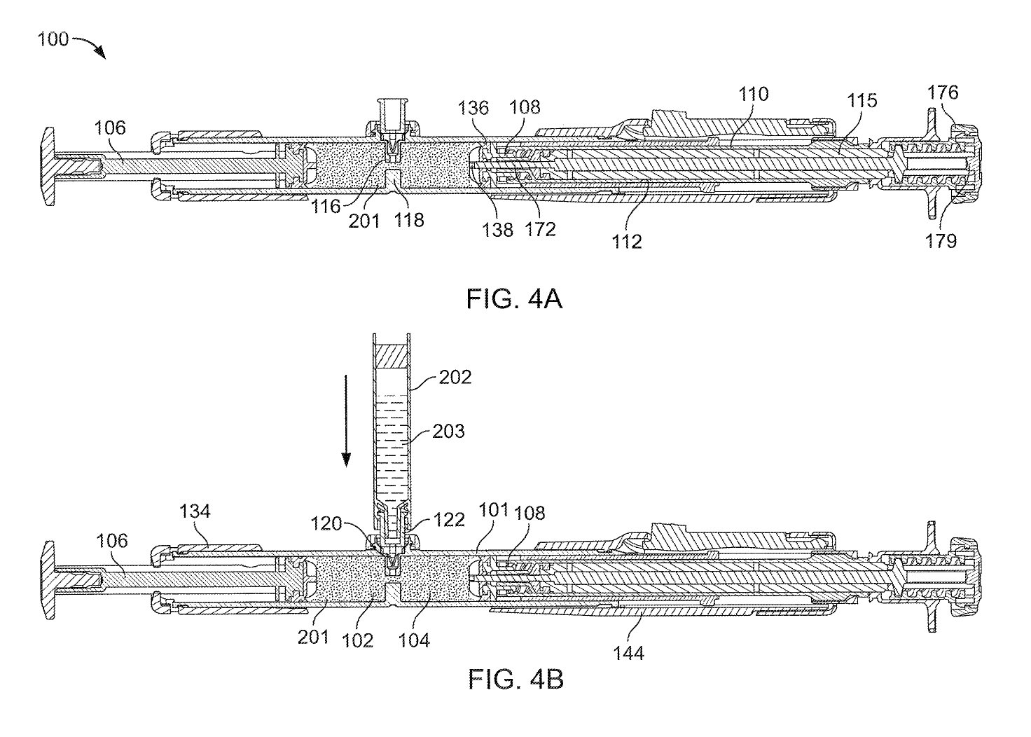

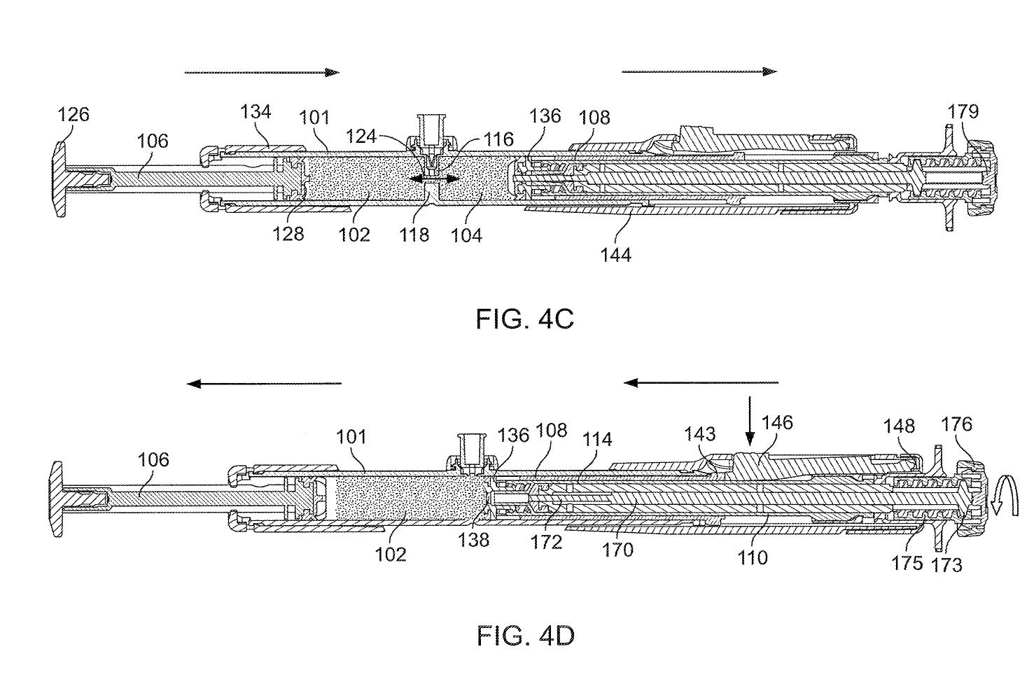

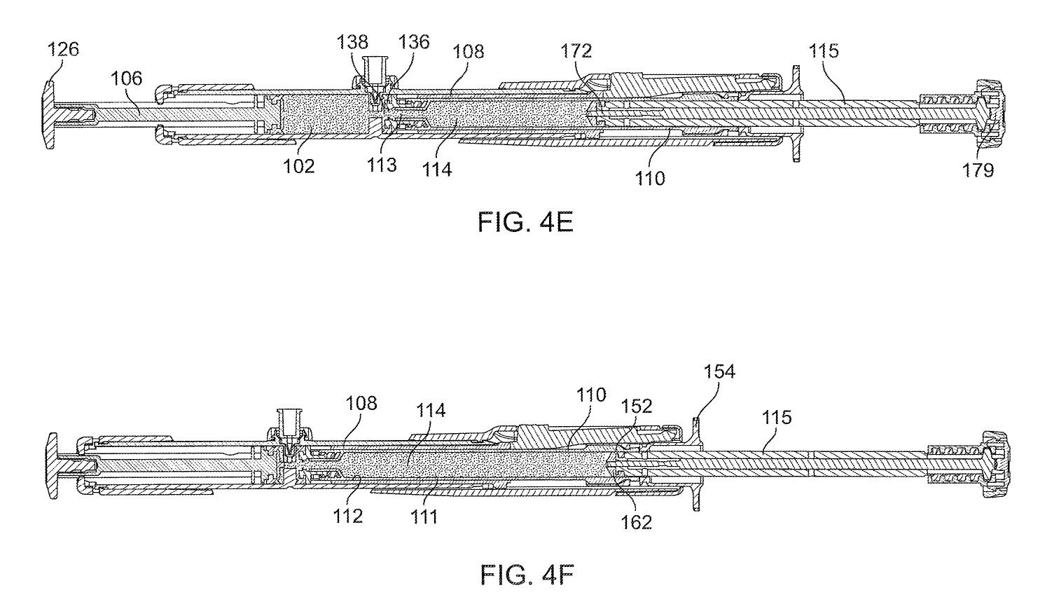

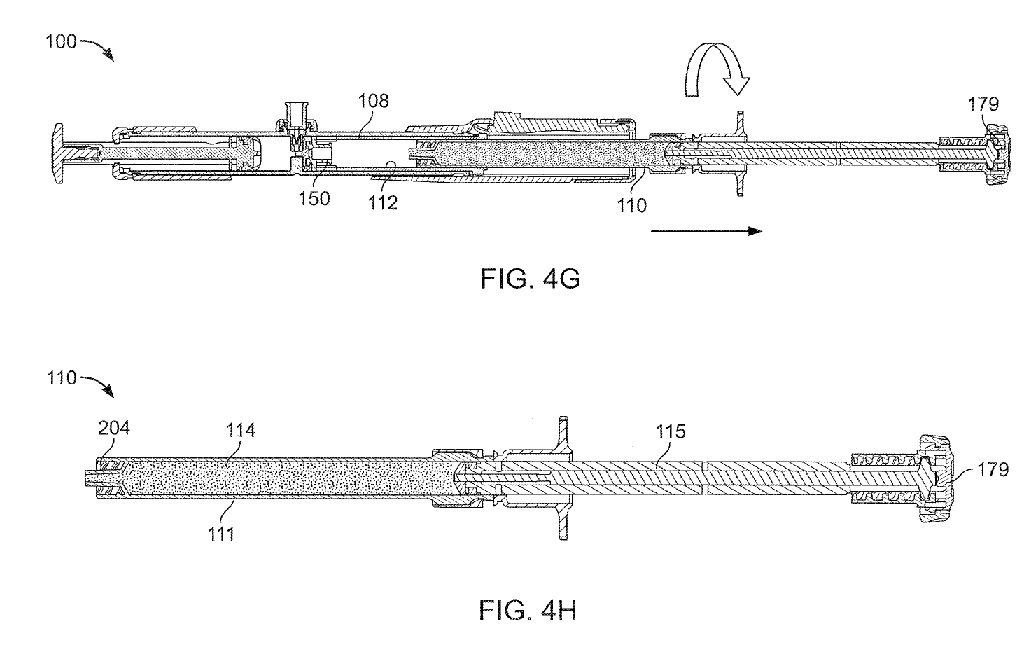

FIGS. 4A-4H illustrate a method of using bone cement mixing system 100. As shown in FIG. 4A, in an initial configuration, bone cement delivery device 110 is disposed within axial bore 112 of second piston 108, and pin 172 is in its fully forward position, sealing off aperture 138 in seal 136. CPM powder 201 is substantially evenly distributed on either side of mixing post 116 and mixing anvil 118.

Referring to FIG. 4B, a liquid injection device (e.g., a syringe) 202 filled with a liquid solution (e.g., a solution of rhBMP-2) 203 is attached to inlet fitting 122, and solution 203 is injected into first and second mixing chambers 102, 104 via inlet fitting 122. Solution 203 passes through one-way valve 120 into CPM powder 201. Solution 203 can be of a strength desired for a particular application. In some embodiments, a small amount of air (e.g., approximately about 0.5 ml of air) is included in liquid injection device 202 and injected into bone cement mixing system 100 after solution 203 to ensure that substantially all liquid is cleared from inlet fitting 122 and one-way valve 120. The combination of solution 203 and CPM powder 201 forms a bone cement paste. After injecting solution 203 into CPM powder 201, liquid injection device 202 can be detached from inlet fitting 122. A cap can then be secured inlet fitting 122 to help prevent the bone cement paste and resulting gases from escaping first and second mixing chambers 102, 104.

By injecting solution 203 and air into the sealed bone cement mixing system 100, an internal pressure is created in mixing chambers 102 and 104, tending to force pistons 106 and 108 outward (e.g., toward opposite ends of housing 101), as shown in FIG. 4C. In addition, the sodium bicarbonate content of CPM powder 201 can generate carbon dioxide when wetted by solution 203, further increasing the internal pressure within mixing chambers 102 and 104. Pistons 106 and 108 can, for example, move outward under gas pressure until resisted by cap 134 and extended end cap 144, respectively. In embodiments in which bone cement mixing system 100 is provided in a retainer tray, the form of the tray can prevent outward movement of pistons 106 and 108. After injection of solution 203 and air, bone cement mixing system 100 can be removed from the retainer tray, allowing pistons 106 and 108 to move further outward as a result of the internal pressure within mixing chambers 102 and 104.

Referring to FIG. 4C, after injecting solution 203 into CPM powder 201, the user presses alternately on head 126 and retainer cap 179, causing first and second pistons 106, 108 to move in an alternating fashion within first and second chambers 102, 104. As a result, the body of wetted powder is forced back and forth past mixing post 116 and mixing anvil 118, thereby initiating the first stage of mixing. During the initial phases of the first stage of mixing, solution 203 is not thoroughly blended with CPM powder 201. However, the relatively large cross-sectional area of passage 124 can allow the user to pass the relatively dry bone cement paste back and forth between mixing chambers 102 and 104 without excessive physical effort. Initially, seals 128 and 136 may be unable to move fully towards the center of housing 101 due to a mass of unmixed paste trapped between seals 128, 136 and mixing post 116 and mixing anvil 118. However, after a number of strokes of first and second pistons 106, 108, full travel can be achieved. At this point, the user can carry out a further set number of full strokes of each piston to complete first stage mixing. For example, upon achieving the full range of travel with first and second pistons 106, 108, the user can complete ten full strokes on each piston to complete the first stage of mixing. Pistons 106 and 108 can be actuated at a rate of about 0.5 stroke per second to about one stroke per second. Generally, as the actuation rate increases, the level of shear experienced within the wetted powder increases. Upon completing the first stage of mixing, second piston 108 is in the fully ‘in’ position and first piston 106 is in the fully ‘out’ position, as shown in FIG. 4D.

Referring to FIG. 4D, upon completion of the first stage of mixing and with second piston 108 in the fully ‘in’ position, the user presses on lever 146 to axially fix second piston 108 relative to housing 101. Lever 146 pivots inward around platform 148 and snaps into a fixed inward position behind block 143, thereby retaining second piston 108 in the fully ‘in’ position. The user then rotates rotary cap 176 to draw plunger shaft 170 and pin 172 outward. Typically, the user will rotate rotary cap 176 one to two full turns, until lugs 173 of plunger shaft 170 come into contact with the end of thread 175. As lugs 173 contact the end of thread 175, further rotation of rotary cap 176 will be prevented. Rotating rotary cap 176 as described causes pin 172 to be removed from aperture 138 of seal 136. As a result, mixing/delivery chamber 114 of bone cement delivery device 110 is placed in fluid communication with first mixing chamber 102. Thus, the increased rotational resistance caused by lugs 173 contacting the end of thread 175 can serve as an indication to the user that fluid communication between first mixing chamber 102 and mixing/delivery chamber 114 has been achieved.

Referring to FIG. 4E, after withdrawing pin 172 from aperture 138 of seal 136, the user again presses head 126 and retainer cap 179 alternately to carry out the second stage of mixing. With second piston 108 restrained against axial movement, plunger 115 is free to slide axially back and forth within mixing/delivery chamber 114 as head 126 and retainer cap 179 are alternately actuated during this second stage of the mixing process. Thus, alternately pressing head 126 and retainer cap 179 causes first piston 106 to slide back and forth within first mixing chamber 102 and causes plunger 115 to slide back and forth within mixing/delivery chamber 114 of bone cement delivery device 110. As a result, the bone cement paste is sequentially forced into and out of mixing/delivery chamber 114 via aperture 138 in seal 136 and a passage formed in reduced diameter tip 113 of bone cement delivery device 110. The passage in tip 113 can have a diameter that is substantially equal to the diameter of aperture 138. The user can carry out a set number of full strokes of first piston 106 and plunger 115 to complete the second stage of mixing. For example, the user can complete ten strokes on piston 106 and plunger 115 to complete the second stage of mixing. In some embodiments, piston 106 and plunger 115 are actuated at a rate of about 0.5 stroke per second to about one stroke per second. The bone cement paste can be passed through aperture 138 at a rate of about 1 ml per second to about 20 ml per second (e.g., about 1.5 ml per second to about 7 ml per second). Upon completing the second stage of mixing, plunger 115 is disposed in the fully ‘out’ position such that substantially all of the bone cement paste is disposed in mixing/delivery chamber 114 of bone cement delivery device 110, as shown in FIG. 4F.

An increased level of shear is created within the bone cement paste during the second stage of mixing as compared to the first stage of mixing because the flow areas of aperture 138 in seal 136 and the passage in tip 113 are substantially smaller than the flow area of passage 124. The flow area of aperture 138 can, for example, be about 90 percent to about 95 percent less than the flow area of passage 124. As the bone cement paste is being passed from the relatively large diameter of first mixing chamber 102 to the relatively small diameters of aperture 138 in seal 136 and the passage formed in tip 113, shear levels within the bone cement paste increase substantially. The level of shear can also be increased by increasing the actuation rate of piston 106 and plunger 115.

Referring to FIG. 4F, after completing the second stage of mixing, the user pulls plunger 115 fully back into extension cap 154, causing seal 162 to be positioned adjacent axial grooves 152 formed on the inner surface of body portion 111 of bone cement delivery device 110. As a result, gas is allowed to pass through axial grooves 152. This vents excess gas pressure in bone cement mixing system 100 to reduce or minimize loss of the bone cement paste by premature ejection when bone cement delivery device 110 is removed from axial bore 112 of second piston 108.

Referring to FIG. 4G, after venting excess gas from bone cement delivery device 110, the user rotates bone cement delivery device 110 counterclockwise (as viewed from the end of retainer cap 179) to release bone cement delivery device 110 from the remainder of bone cement mixing system 100. Rotating bone cement delivery device 110 can, for example, release bone cement delivery device 110 from the lock provided by Luer Lock taper 150 of second piston 108. Upon releasing the connection between bone cement delivery device 110 and second piston 108, bone cement delivery device 110 is removed from axial bore 12 of second piston 108.

As shown in FIG. 4H, bone cement delivery device 110 carries a standard Luer Lock fitting 204 at its distal end. After removing bone cement delivery device 110 from axial bore 112 of second piston 108, Luer Lock fitting 204 can be connected to an appropriate needle (not shown), and the combination of bone cement delivery device 110 and the needle can be used to inject the bone cement paste into a treatment site (e.g., a bone fracture site) in a patient. To inject the bone cement paste, the user can depress retainer cap 179 to axially displace plunger 115, causing the bone cement paste to be expelled from mixing/delivery chamber 114 through an opening at the distal end of bone cement delivery device 110. After injecting the bone cement paste into the patient, bone cement mixing system 100, including detached bone cement delivery device 110, can be discarded.

While certain embodiments have been described, other embodiments are possible.

As an example, while bone cement delivery device 110 has been described as being secured to second piston 108 using a Luer Lock fitting, other techniques can be used to secure, bone cement delivery device 110 to second piston 108. Examples of other structures that can be used to secure bone cement delivery device 110 to second piston 108 include. Luer tapers, O-ring sealed connections, olive fittings, and threaded taper fittings.

As another example, while tubular body portion 111 of bone cement delivery device 110 has been described as including axial grooves on its inner surface to allow excess gas to be vented from mixing/delivery chamber 114, other arrangements can alternatively or additionally be used to vent excess gas. In some embodiments, for example, the tubular body portion of the bone cement delivery device includes one or more apertures that are covered by a porous membrane configured to allow gases, but not liquids, to pass therethrough.

As an additional example, while pin 172 has been described as being extended from plunger 115 by rotating rotary cap 176, other arrangements can alternatively or additionally be used to allow pin 172 to be extended from plunger 115. In certain embodiments, for example, the user can simply push and pull plunger shaft 170 and pin 172 axially to extend, pin 172 from the end of the plunger and to retract pin 172 into the plunger. In such embodiments, the plunger shaft can include projections that releasably engage apertures formed in the plunger (or vice versa) in order to lock the pin in the extended and retracted positions.

As a further example, while embodiments described above include a hollow plunger with a plunger shaft and pin disposed therein, in some embodiments, the plunger is a solid member. In such embodiments, for example, the bone cement mixing system can be used without positioning a pin in the seal of the second piston during the first stage of mixing.

As an additional example, while the embodiments discussed above include a lever that can be manipulated to axially fix second piston 108 relative to housing 101, other arrangements are possible. In some embodiments, for example, a ring including projects that extend radially inward from its inner surface is threadedly coupled to an end region of the housing. The ring can be configured such that second piston is allowed to slide axially therethrough when ring is in an unscrewed position and the second piston is prevented from moving axially relative to the ring and the housing when the ring is in a screwed in position. In the screwed in position, the projection extending from the inner surface of the ring can contact the block extending from the outer surface of the second piston, thereby fixing the second piston in an axially position relative to the housing.

As another example, while bone cement delivery device 110 has been described as being disposed within axial bore 112 of second piston 108, other arrangements are possible. In some embodiments, the bone cement delivery device is secured to a fluid fitting extending from an outer surface of housing 101. In certain embodiments, for example, the bone cement delivery device can be secured to the same fluid fitting to which liquid injection device 202 is secured in order to inject solution 203 into CPM powder 201. Bone cement delivery device 110 can alternatively or additionally be secured to an additional fluid fitting extending from the housing. In some embodiments, the bone cement mixing system (e.g., the fluid fitting of the bone cement delivery device) includes a valve that can be moved to a first position to place the bone cement delivery device in fluid communication with first mixing chamber 102 and can be moved to a second position to fluidly disconnect the bone cement delivery device from first mixing chamber 102. In such embodiments, for example, the valve can be closed to prevent fluid communication between the bone cement delivery device and first mixing chamber 102 during the first stage of mixing, and the valve can be opened to allow fluid communication between the bone cement delivery device and first mixing chamber 102 during the second stage of mixing. The valve can similarly be configured to selectively open and close fluid communication between first mixing chamber 102 and second mixing chamber 104 so that first and second mixing chambers 102 and 104 can be fluidly connected to one another during the first stage of mixing and can be fluidly disconnected from one another during the second stage of mixing.

As a further example, while liquid injection device 202 has been described as a traditional syringe, other types of liquid injection devices can alternatively or additionally be used. For example, syringe pumps, screw pumps, peristaltic pumps, and/or pre-pressurized containers can be used.

As a further example, while the bone cement powder has been described as CPM powder, one or more other types of bone cement powder can alternatively or additionally be used. Examples of bone cement powders include calcium phosphate based powders and polymethyl methacrylate based powders. Any of various osteoconductive powders, such as ceramics, calcium sulfate or calcium phosphate compounds, hydroxyapatite, deproteinized bone, corals, and certain polymers, can alternatively or additionally be used.

As an additional example, while solution 203 has been described as a solution of rhBMP-2, one or more other solutions can alternatively or additionally be used. Examples of other solutions include aqueous-based solutions, such as saline and phosphate buffered saline (PBS). In certain embodiments, the liquid has a PH level of about 4.0 to about 8.0. Another example of a solution that is used in certain embodiments is methyl methacrylate monomer.

While certain embodiments discussed above include the use of rhBMP-2, any of various other active agents can alternatively or additionally be used. The active agent of the bone cement paste can, for example, be selected from the family of proteins known as the transforming growth factor-beta (TGF-ÿ) superfamily of proteins, which includes the activins, inhibins, and bone morphogenetic proteins (BMPs). In some embodiments, the active agent includes at least one protein selected from the subclass of proteins known generally as BMPs. BMPs have been shown to possess a wide range of growth and differentiation activities, including induction of the growth and differentiation of bone, connective, kidney, heart, and neuronal tissues. See, for example, descriptions of BMPs in the following publications: BMP-2, BMP-3, BMP-4, BMP-5, BMP-6, and BMP-7 (disclosed, for example, in U.S. Pat. Nos. 5,013,649 (BMP-2 and BMP-4); 5,116,738 (BMP-3); 5,106,748 (BMP-5); 5,187,076 (BMP-6); and 5,141,905 (BMP-7)); BMP-8 (disclosed in PCT WO 91/18098); BMP-9 (disclosed in PCT WO 93/00432); BMP-10 (disclosed in PCT WO 94/26893); BMP-11 (disclosed in PCT WO 94/26892); BMP-12 and BMP-13 (disclosed in PCTWO 95/16035); BMP-15 (disclosed in U.S. Pat. No. 5,635,372); BMP-16 (disclosed in U.S. Pat. No. 6,331,612); MP52/GDF-5 (disclosed in PCT WO 93/16099); and BMP-17 and BMP-18 (disclosed in U.S. Pat. No. 6,027,917). Other TGF-ÿ proteins that may be useful as the active agent of the bone cement paste include Vgr-2 and any of the growth and differentiation factors (GDFs).

A subset of BMPs that may be used in certain embodiments includes BMP-2, BMP-4, BMP-5, BMP-6, BMP-7, BMP-8, BMP-9, BMP-10, BMP-11, BMP-12 and BMP-13. In some embodiments, the composition contains two or more active agents (e.g., BMP-2 and BMP-4). Other BMPs and TGF-ÿ proteins may also be used.

The active agent may be recombinantly produced, or purified from another source. The active agent, if a TGF-ÿ protein such as a BMP, or other dimeric protein, may be homodimeric, or may be heterodimeric with other BMPs (e.g., a heterodimer composed of one monomer each of BMP-2 and BMP-6) or with other members of the TGF-ÿ superfamily, such as activins, inhibins and TGF-ÿ (e.g., a heterodimer composed of one monomer each of a BMP and a related member of the TGF-ÿ superfamily). Examples of such heterodimeric proteins are described, for example in published PCT Patent Application WO 93/09229.

Other embodiments are in the claims.

What is claimed is:

1. A bone cement mixing system, comprising:
a housing defining a first chamber, a second chamber, and a passage fluidly connecting the first and second chambers;
a first piston slidably disposed within the first chamber;
a second piston slidably disposed within the second chamber; and
a bone cement delivery device disposed within the second chamber, the bone cement delivery device defining a third chamber and being adaptable to place the third chamber in fluid communication with the first chamber.

2. The bone cement mixing system of claim 1, wherein the bone cement delivery device is disposed within a bore in the second piston.

3. The bone cement mixing system of claim 2, wherein the bone cement delivery device comprises an axially displaceable pin, the pin being arranged to fit within an aperture in a seal of the second piston such that there is substantially no fluid communication between the first chamber of the housing and the third chamber of the bone cement delivery device when the pin is disposed within the bore in the seal of the second piston.

4. The bone cement mixing system of claim 3, wherein the pin is capable of being retracted from the aperture in the seal of the second piston, the first chamber of the housing being in fluid communication with the third chamber of the bone cement delivery device when the pin is retracted from the aperture in the seal of the second piston.

5. The bone cement mixing system of claim 1, wherein the bone cement delivery device is slidably disposed within the second chamber.

6. The bone cement mixing system of claim 1, wherein the bone cement delivery device comprises a syringe.

7. The bone cement mixing system of claim 6, wherein the syringe comprises a fitting configured to secure the syringe to the second piston.

8. The bone cement mixing system of claim 7, wherein the fitting comprises a Luer Lock fitting.

9. The bone cement mixing system of claim 1, wherein the bone cement delivery device comprises a tube and circumferentially spaced ribs extending from the tube, the circumferentially spaced ribs arranged to cooperate with the second piston to form channels configured to permit gases to pass therethrough.

10. The bone cement mixing system of claim 1, wherein the passage that fluidly connects the first and second chambers has a reduced cross-sectional area relative to the first and second chambers.

11. The bone cement mixing system of claim 10, wherein a passage that fluidly connects the first and third chambers when the third chamber is placed in fluid communication with the first chamber has a reduced cross-sectional area relative to the first and third chambers.

12. The bone cement mixing system of claim 1, further comprising a bone cement powder disposed within at least one of the first and second chambers.

13. The bone cement mixing system of claim 12, wherein the bone cement powder comprises osteoconductive powder.

14. The bone cement mixing system of claim 13, wherein the bone cement powder comprises calcium phosphate/sodium bicarbonate blended powder.

15. The bone cement mixing system of claim 12, wherein the bone cement powder forms a bone cement paste when a liquid is added to the bone cement powder.

16. The bone cement mixing system of claim 15, wherein the bone cement paste can be mixed by axially displacing the first and second pistons within the first and second chambers, respectively.

17. The bone cement mixing system of claim 15, wherein the liquid comprises a solution of recombinant human bone morphogenetic protein.

18. The bone cement mixing system of claim 1, wherein the passage that fluidly connects the first and second chambers is partially defined by a mixing post and a mixing anvil extending from an inner surface of the housing.

19. The bone cement mixing system of claim 1, wherein the housing comprises an inlet fitting configured to allow a liquid injection device to be secured thereto.

20. The bone cement mixing system of claim 19, wherein the liquid injection device is in fluid communication with the first and second chambers when secured to the inlet fitting.

21. The bone cement mixing system of claim 20, wherein the bone cement mixing system is a single-use-bone cement mixing system.

22. A system, comprising:
a housing defining a first chamber, a second chamber, and a passage fluidly connecting the first and second chambers, the housing being configured so that a liquid injection device can be secured thereto, the liquid injection device being in fluid communication with at least one of the first and second chambers when secured to the housing;
a first piston slidably disposed within the first chamber;
a second piston slidably disposed within the second chamber; and
a bone cement delivery device disposed in the second chamber.

23. The system of claim 22, wherein the bone cement delivery device is disposed within a bore in the second piston.

24. The system of claim 22, further comprising a liquid injection device secured to the housing.

25. The system of claim 24, further comprising a bone cement powder disposed within at least one of the first and second chambers, wherein the bone cement powder forms a bone cement paste when a liquid is transferred from the liquid injection device into the at least one of the first and second chambers.

26. The system of claim 22, wherein the first and second pistons are capable of passing bone cement paste back and forth between the first and second chambers when the first and second pistons are alternately depressed.

27. The system of claim 22, wherein the bone cement delivery device defines a third chamber, the bone cement delivery device being adaptable to place the third chamber in fluid communication with the first chamber.

28. The system of claim 27, wherein the first piston and a plunger of the bone cement delivery device are capable of passing bone cement paste back and forth between the first and third chambers when the first piston and the plunger are alternately depressed and the third chamber of the bone cement delivery device is in fluid communication with the first chamber.

29. The system of claim 28, wherein the system is a single-use system.

30. A method, comprising:
passing a bone cement paste through a first passage that fluidly connects a first chamber and a second chamber, the first passage configured to cause a first level of shear within the bone cement paste as the bone cement paste is passed therethrough; and
passing the bone cement paste through a second passage that fluidly connects the first chamber to a third chamber, the second passage configured to cause a second level of shear within the bone cement paste as the bone cement paste is passed therethrough, the second level of shear being different than the first level of shear.

31. The method of claim 30, wherein the third chamber is formed by a bone cement delivery device disposed in the second chamber.

32. The method of claim 31, further comprising removing the bone cement delivery device from the second chamber after passing the bone cement paste into the third chamber.

33. The method of claim 30, wherein passing the bone cement paste through the first passage imparts a first level of shear to the bone cement paste and passing the bone cement paste through the second passage imparts a second level of shear to the bone cement paste, the first level of shear being lower than the second level of shear.

34. The method of claim 33, wherein the method comprises passing the bone cement paste through the first passage prior to passing the bone cement paste through the second passage.

35. The method of claim 30, further comprising introducing a liquid into at least one of the first and second chambers.

36. The method of claim 35, wherein the liquid comprises a solution of recombinant human bone morphogenetic protein.
